(19) United States
(12) Patent Application Publication
(10) Pub. No.: US 2022/0233089 A1
(43) Pub. Date: Jul. 28, 2022

(54) TECHNIQUES FOR DETERMINING TISSUE TYPES

(71) Applicant: NOVASCAN, INC., Milwaukee, WI (US)

(72) Inventors: Les BOGDANOWICZ, Park Ridge, IL (US); Anthony APPLING, Crestwood, KY (US); Donato M. CERES, Chicago, IL (US); Terry DAGLOW, Allen, TX (US); Alexander GRYCUK, Mt. Prospect, IL (US); Benjamin MORRIS, Jeffersonville, IN (US); Isaac RAIJMAN, Houston, TX (US); Christen Andrew SPRINGS, Sunrise Beach, TX (US); Paul Richard VOITH, Cedarburg, WI (US)

(21) Appl. No.: 17/695,745

(22) Filed: Mar. 15, 2022

Related U.S. Application Data
(63) Continuation-in-part of application No. 17/397,896, filed on Aug. 9, 2021.
Application No. 17/397,896 is a continuation-in-part of application No. 17/412,973, filed on Aug. 26, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.

(51) Int. Cl. A61B 5/0537; A61B 5/00

(52) U.S. Cl. CPC A61B 5/0537; A61B 5/6851; A61B 2562/046; A61B 2562/164; A61B 2562/043; A61B 5/6853

(57) ABSTRACT

In various embodiments, a medical device includes an instrument head that includes two or more electrodes and a medical device tool, an impedance bridge selectively coupled to the two or more electrodes, and a processor coupled to the impedance bridge. In various embodiments, a method for controlling medical device tools comprises recording, at one or more frequencies, two or more impedance measurements, wherein each impedance measurement is associated with two or more electrodes included in an instrument head of a medical device; and determining, based on the two or more impedance measurements, a tissue type map at a location associated with the instrument head.

### CROSS-REFERENCES TO RELATED APPLICATIONS

The present application is a continuation-in-part of the U.S. patent application titled, “IMPEDANCE-CALIBRATED DIAGNOSTIC MEDICAL DEVICES,” filed on Aug. 9, 2021, and having Ser. No. 17/397,896, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021. The present application is also a continuation-in-part of the U.S. patent application titled, “TECHNIQUES FOR CONTROLLING MEDICAL DEVICE TOOLS,” filed on Aug. 26, 2021, and having Ser. No. 17/412,973, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021. The subject matter of these related applications is hereby incorporated herein by reference.

### BACKGROUND

### Field of the Various Embodiments

Embodiments of the present disclosure relate generally to electronics and medical diagnostic technology and, more specifically, to techniques for determining tissue types.

### Description of the Related Art

In minimally invasive medical procedures, a healthcare professional typically inserts a medical device, such as an endoscope or a bronchoscope, into the patient's body and positions the instrument head of the medical device at a target location, such as the location of a tumor. The instrument head usually includes some form of tool, such as and without limitation, a camera, a fiber optic light source, a pair of forceps, and/or a tissue sample extraction tool that can be used to extract tissue samples from the target location for further evaluation.

One drawback that exists with many conventional medical devices is the difficulty of determining a tissue type at a location where the instrument head of a medical device tool is positioned. For example, a healthcare professional can visually inspect an image of tissue captured by a camera while the tissue is illuminated by a light source. However, tissue types can vary in appearance, and different tissue types can have similar appearances. As another example, the tissue type on one side of the instrument head, such as on a left side of the instrument head, can differ from the tissue type on the other side of the instrument head, such as on a right side of the instrument head. As yet another example, a medical device can be used to extract a tissue sample from a given location, and a healthcare professional can determine how to treat the tissue at the given location by visually inspecting or performing a biopsy on the tissue sample. However, if the instrument head moves between sampling the tissue and treating the tissue, then the healthcare professional could end up treating tissue that is different than the extracted tissue.

In view of the above drawbacks, medical devices oftentimes include components that are configured to determine the tissue type contacting the instrument head. However, many techniques for determining tissue type are inaccurate and, accordingly, are insufficient for confirming that a given tool is positioned correctly at a given target location. For example, triangulation and ultrasound imaging typically require calibrating the relevant positioning system relative to both the medical device tool and a mapping of the patient's body via a medical scan. Errors introduced in the calibration process can produce errors when determining whether the medical device tool is positioned correctly at the target location. Also, any physiological changes within the patient, such as the size, shape, or location of a tumor, between the time when a medical scan is conducted and the time when the medical procedure begins can change the target location. Thus, positioning a medical device tool based on a medical scan can sometimes result in applying the medical device tool to healthy tissue instead of at the target location.

As the foregoing illustrates, what is needed in the art are more effective techniques for determining tissue types at locations where the instrument heads of medical devices are positioned.

### SUMMARY

Embodiments are disclosed for medical devices. In various embodiments, a medical device includes an instrument head that includes two or more electrodes and a medical device tool; an impedance bridge selectively coupled to the two or more electrodes; and a processor coupled to the impedance bridge.

Embodiments are disclosed for controlling a medical device. In various embodiments, a method includes controlling a medical device includes recording, at one or more frequencies, two or more impedance measurements, each impedance measurement being associated with two or more electrodes included in an instrument head of the medical device, and determining, based on the two or more impedance measurements, a map of tissue types at a location associated with the instrument head.

At least one technical advantage of the disclosed medical device relative to the prior art is that the disclosed medical device is able to determine a map of tissue types at a location where the instrument head of a medical device is positioned prior to when a medical device tool is activated or during activation. For example, the disclosed medical device can determine whether the tissue types of portions of tissue located where the instrument head of a medical device is positioned match the expected tissue types at a given target location prior to activating the relevant medical device tool. In this manner, the disclosed medical device can ensure that medical device tools are applied to a selected tissue type, such as a tumor, rather than some other tissue type, such as healthy tissue. Also, the disclosed medical instrument can apply medical device tools at target locations more accurately than is possible with conventional medical devices. Consequently, the disclosed medical device can be used to perform various procedures, such as and without limitation, delivering therapeutic drugs or energy or extracting tissue samples, at specific locations more accurately and reliably than what can be achieved using conventional medical devices. These technical advantages provide one or more technological advancements over prior art approaches.

### BRIEF DESCRIPTION OF THE DRAWINGS

FIG. 1 illustrates a medical device, according to various embodiments;

FIG. 2 is a more detailed illustration of the instrument head of FIG. 1, according to various embodiments;

FIG. 3 is a more detailed illustration of the instrument head of FIG. 1, according to other various embodiments;

FIG. 4A is an illustration of an electrode configuration associated with the instrument head of FIG. 3, according to various embodiments;

FIG. 4B is an illustration of an electrode configuration associated with the instrument head of FIG. 3, according to other various embodiments;

FIG. 5 is a more detailed illustration of the instrument head of FIG. 1, according to other various embodiments;

FIG. 6 is a more detailed illustration of the instrument head of FIG. 1, according to other various embodiments;

FIG. 7 is more detailed illustration of the instrument head of FIG. 1, according to other various embodiments;

FIGS. 8A-8B are more detailed illustrations of the instrument head of FIG. 1, according to other various embodiments;

FIG. 9 is a more detailed illustration of the instrument head of FIG. 1, according to other various embodiments;

FIG. 10 is a more detailed illustration of the instrument head of FIG. 1, according to other various embodiments;

FIG. 11 is a more detailed illustration of the instrument head of FIG. 1, according to other various embodiments;

FIG. 12 is a more detailed illustration of the instrument head of FIG. 1, according to other various embodiments;

FIG. 13 is a more detailed illustration of the instrument head of FIG. 1, according to other various embodiments;

FIG. 14 is a more detailed illustration of the instrument head of FIG. 1, according to other various embodiments;

FIGS. 15A-15B are more detailed illustrations of the instrument head of FIG. 1, according to other various embodiments;

FIGS. 16A-16B are more detailed illustrations of the instrument head of FIG. 1, according to other various embodiments;

FIGS. 17A-17B are more detailed illustrations of the instrument head of FIG. 1, according to other various embodiments;

FIG. 18A is a more detailed illustration of the instrument head of FIG. 1, according to other various embodiments;

FIG. 18B is an illustration of a grid pattern associated with the instrument head of FIG. 18A, according to various embodiments;

FIG. 19 is a more detailed illustration of the external electrical components of FIG. 1, according to various embodiments;

FIG. 20 is a more detailed illustration of the medical device of FIG. 1, according to various embodiments; and

FIG. 21 is a flow diagram of method steps for controlling a medical device tool, according to various embodiments.

### DETAILED DESCRIPTION

In the following description, numerous specific details are set forth to provide a more thorough understanding of the various embodiments. However, in the range of embodiments of the concepts includes some embodiments omitting one or more of these specific details.

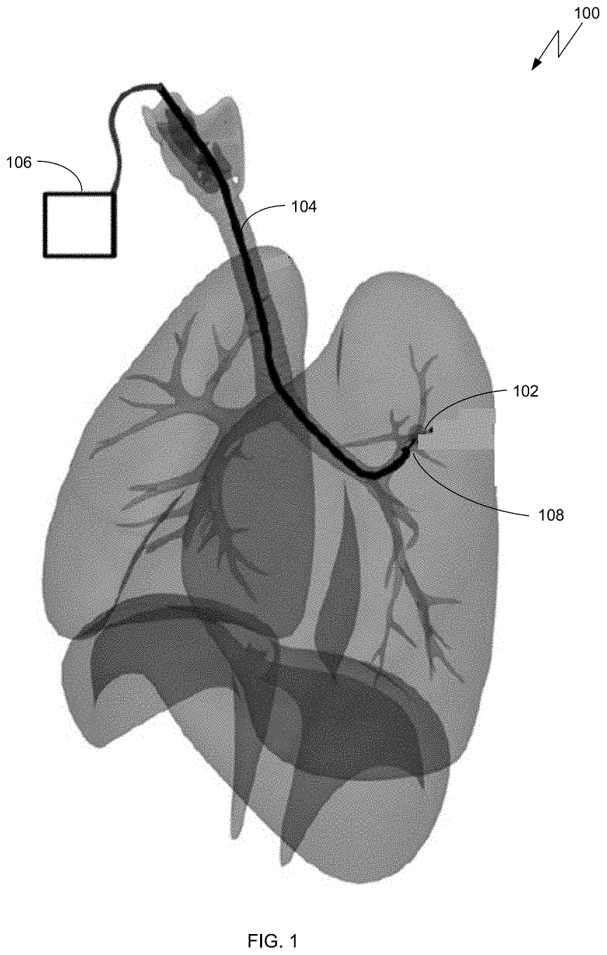

FIG. 1 illustrates a medical device 100, according to various embodiments. As shown, the medical device 100 includes, without limitation, an instrument head 108, wires 104, and external electrical components 106. The instrument head 108 is positioned at a location 102 (e.g., a location of a tumor). While not shown, the instrument head 108 includes, without limitation, two or more electrodes, a conduit, and a medical device tool, such as and without limitation, a camera, a fiber optic light source, a therapeutic drug delivery tool that delivers a therapeutic drug to the location 102, an energy delivery tool that delivers energy to the location 102, or a tissue sample extraction tool that extracts a tissue sample from the location 102 for further evaluation. The external electrical components 106 generate current at various frequencies. The wires 104 conduct the current between the external electrical components 106 and the instrument head 108. The external electrical components 106 include a processor that selectively couples to the two or more electrodes in the instrument head 108. The processor of the external electrical components 106 measures the impedance of current conducted through tissue between the selected electrodes. As described in greater detail below, the medical device 100 generates, based on the impedance measurements, a tissue type map of tissue at the location 102. For example and without limitation, based on the impedance measurements, the tissue type map can indicate whether portions of tissue at the location 102 of the instrument head 108 are a tumor tissue type or a non-tumor tissue type.

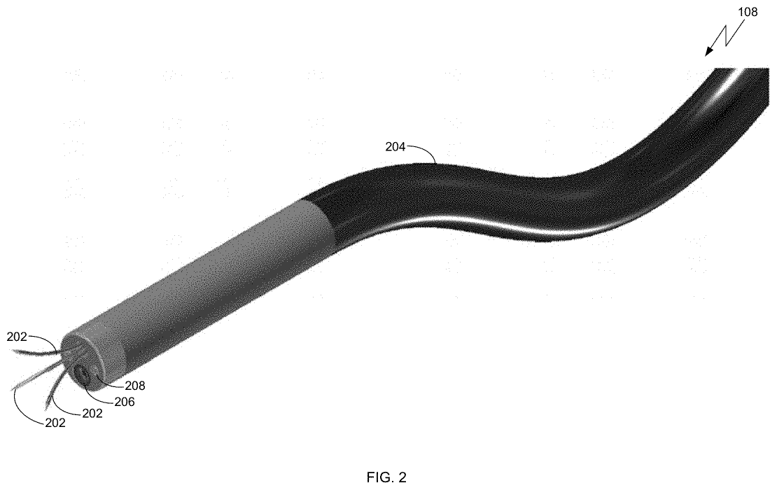

FIG. 2 is a more detailed illustration of the instrument head 108 of FIG. 1, according to various embodiments. As shown, the instrument head 108 includes, without limitation, two or more electrodes 202, a sheath 204 including an aperture 302, a camera 206, and a light source 208.

The sheath 204 encloses wires 104 that couple the two or more electrodes 202 to the external electrical components 106. The wires 104 selectively couple two or more of the electrodes 202 to the external electrical components 106. The selected electrodes 202 conduct current, at various frequencies, through tissue between the selected electrodes 202. Although not shown in FIG. 2, the external electrical components 106 measure an impedance of the current conducted by the selected electrodes 202. The external electrical components 106 can record a set of impedance measurements for different combinations of selected electrodes 202. As an example (without limitation), the external electrical components 106 can record a first impedance measurement of the tissue between two or more electrodes 202 on a left side of the instrument head 108 and a second impedance measurement of the tissue between two or more electrodes 202 on a right side of the instrument head 108. As another example (without limitation), the external electrical components 106 can record a first impedance measurement between two or more electrodes 202 when the instrument head 108 is positioned at a first location and a second impedance measurement between the two or more electrodes 202 when the instrument head 108 is positioned at a second location. For each impedance measurement, the external electrical components 106 determine a tissue type of the tissue between the selected two or more electrodes 202. The external electrical components 106 generate a tissue type map based on the impedance measurements.

The sheath 204 also encloses wires that couple the camera 206 and the light source 208 to the external electrical components 106 (e.g., a power source, a processor, a display, or the like). The sheath 204 physically protects the enclosed wires from contact with an interior surface of a catheter. The sheath 204 also electrically insulates the enclosed wires while conducting current, which preserves the integrity of an electrical signal carried by the current and prevents the current from being conducted through other parts of the catheter or tissue contacting the sheath 204.

In some embodiments, the external electrical components 106 can perform one or more operations, based on the tissue type map, to control one or both of the camera 206 or the light source 208. For example (without limitation), if the tissue type map indicates that tissue at a location associated with the instrument head 108 is of a tumor tissue type, the external electrical components 106 can activate the camera 206 to capture an image of the tissue. In various embodiments, the external electrical components 106 can store the captured image and/or display the captured image on a display for viewing by a healthcare professional. If the tissue type map indicates that tissue at a location associated with the instrument head 108 is of a non-tumor tissue type, the external electrical components 106 can refrain from activating the camera. Selectively activating the camera 206 based on the tissue type map can cause the medical device to limit captured images to tissue of the tumor tissue type. As another example (without limitation), if the tissue type map indicates that tissue at a location associated with the instrument head 108 is of a tumor tissue type, the external electrical components 106 can deliver power to the light source 208 to illuminate the tissue between the electrodes. If the tissue type map indicates that tissue at a location associated with the instrument head 108 is of a non-tumor tissue type, the external electrical components 106 can refrain from delivering power to the light source 208. Selectively powering the light source 208 based on the tissue type map can identify, for a healthcare professional, the tissue of the tumor tissue type.

As shown, the electrodes 202 of the instrument head 108 have a curved shape. In some embodiments, each of the two or more electrodes 202 includes a flexible material, such as aluminum. For example (without limitation), in some embodiments, each of the two or more electrodes 202 bends or curves when extended from the aperture 302 and straightens when retracted into the aperture 302. In some embodiments, each of the two or more electrodes includes a shape memory material, such as Nitinol, which causes the electrodes to form a particular shape (e.g., a curved shape) when the electrode is in an unconstrained state (e.g., when the electrode is extended from the aperture 302).

Although not shown in FIG. 2, in various embodiments, the instrument head 108 includes other types of medical device tools. For example (without limitation), the instrument head 108 can include a conduit that delivers therapeutic drugs or energy and/or a tissue extractor that extracts a tissue sample of the tissue at the location associated with the instrument head 108. The external electrical components 106 can perform one or more operations, based on the tissue type map, to control these and other types of medical device tools included in the instrument head 108. Using the tissue type map to perform the one or more operations can operations can allow a healthcare professional to deliver therapeutic drugs or energy selectively to tissue of a particular tissue type and/or to extract a tissue sample of a particular tissue type for further evaluation.

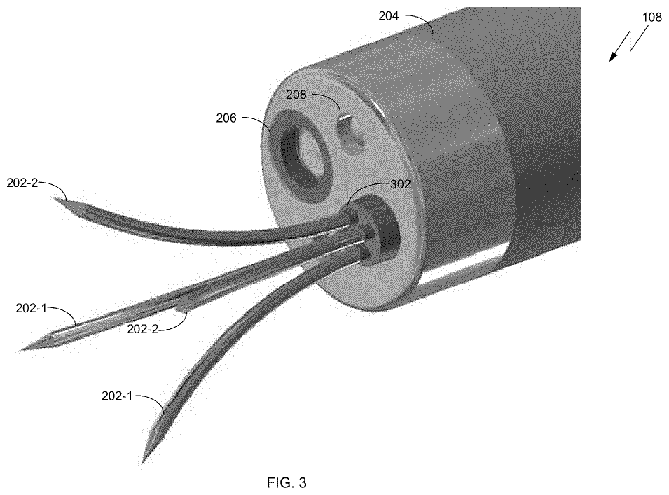

FIG. 3 is a more detailed illustration of the instrument head of FIG. 1, according to other various embodiments. As shown, the instrument head 108 includes, without limitation, two electrode pairs 202-1, 202-2, an aperture 302, a sheath 204, a camera 206, and a light source 208. As previously discussed, the wires 104 selectively couple two or more of the electrodes 202 to the external electrical components 106. For example (without limitation), when the external electrical components 106 are coupled to only a first electrode pair 202-1 that is positioned on a left side of the instrument head 108, the external electrical components 106 record impedance measurements that indicate a tissue type located on a left side of the instrument head 108. When the external electrical components 106 are coupled to only a second electrode pair 202-2 that is positioned on a right side of the instrument head 108, the external electrical components 106 record impedance measurements that indicate a tissue type located on a right side of the instrument head 108. When the external electrical components 106 are coupled to both electrode pairs 202-1, 202-2, the external electrical components 106 record impedance measurements that indicate a tissue type located ahead of (e.g., distal to) the instrument head 108.

As shown, each electrode of the electrode pairs 202-1, 202-2 is shaped to curve outward relative to a longitudinal axis (e.g., a lengthwise axis) of the instrument head 108. In various embodiments, each electrode of the electrode pairs 202-1, 202-2 extends to an adjustable extension length relative to the aperture 302 of the instrument head 108. For example (without limitation), in various embodiments, the external electrical components 106 includes an electrical or mechanical actuator that, when operated, extends one or more of the electrodes from the aperture 302 and/or retracts one or more electrodes toward the aperture 302.

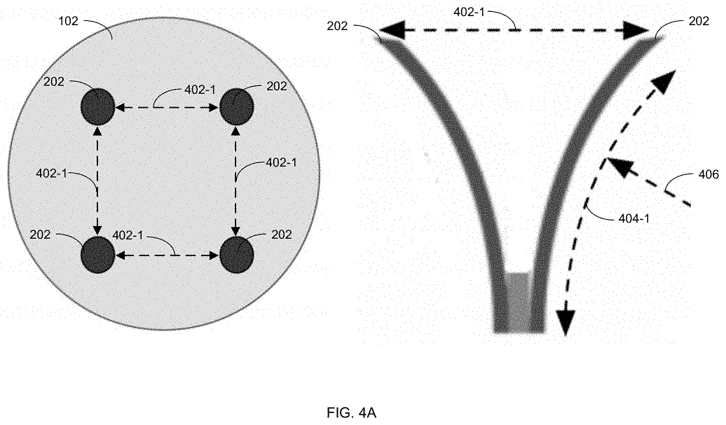

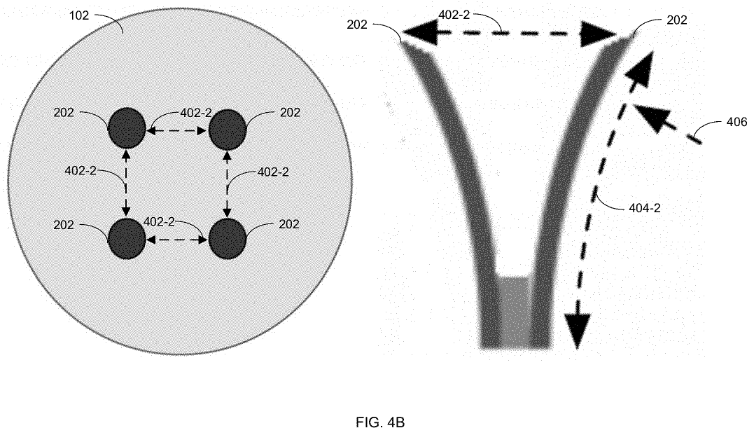

FIG. 4A is an illustration of an electrode configuration associated with the instrument head 108 of FIG. 3, according to various embodiments. As previously discussed, the electrodes of the instrument head can extend from the aperture 302 of the sheath 204 to an adjustable extension length relative to the aperture 302. In FIG. 4A, the electrodes extend from the aperture 302 to an extension length 404-1 that is long. Due to a curvature 406 of the electrodes, a distance 402-1 between each pair of electrodes 202 is large. Due to the large distance 402-1, the impedance measurements by the external electrical components 106 indicate a tissue type of a large portion of tissue at the location 102 of the instrument head 108. The small area results in a coarse, low-resolution determination of the tissue type of a large area of tissue.

FIG. 4B is an illustration of an electrode configuration associated with the instrument head 108 of FIG. 3, according to other various embodiments. As previously discussed, the electrodes of the instrument head can extend from an aperture 302 to a selected length. In FIG. 4B, the electrodes extend, relative to the aperture 302, to an extension length 404-2 that is shorter than the extension length 404-1 shown in FIG. 4A. Due to a curvature 406 of the electrodes, a distance 402-2 between each pair of electrodes 202 is smaller than the distance 402-1 shown in FIG. 4A. Due to the small distance 402-2, the impedance measurements by the external electrical components 106 indicate a tissue type of a small portion of tissue at the location 102 of the instrument head 108. The small area results in a precise, high-resolution determination of the tissue type of a small area of tissue.

In various embodiments, a medical device can adjust the extension lengths 404 of the two or more electrodes 202, relative to the aperture 302, to adjust the impedance measurements during a medical procedure. For example (without limitation), the medical device can initially record impedance measurements while the extension length 404 of the electrodes relative to the aperture 302 is long, and the external electrical components 106 can generate a coarse tissue type map. When the coarse tissue type map indicates that the tissue contacting the instrument head 108 is of a selected tissue type (e.g., a tumor tissue type), the electrodes can retract toward the aperture 302 to a shorter extension length 404 relative to the aperture 302, and the external electrical components 106 can generate a fine tissue type map over a smaller area. By adjusting the extension lengths 404 of the electrodes 202 relative to the aperture 302, the medical device can quickly determine a general area of a selected tissue type based on a low-resolution tissue type map, and then precisely locate the selected tissue type within the general area based on high-resolution tissue type map.

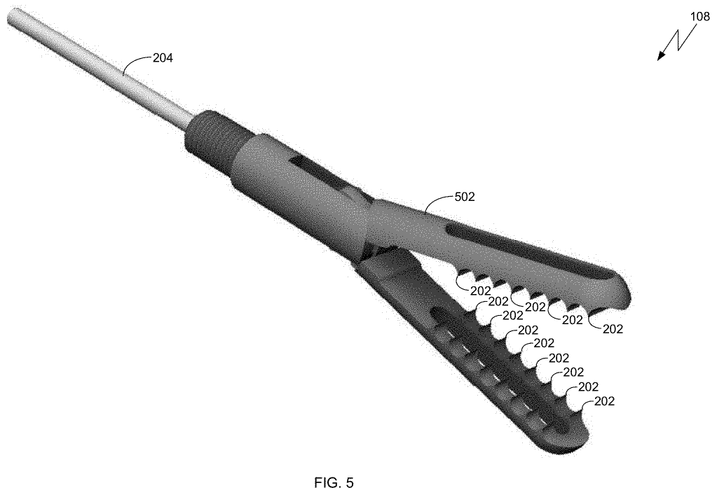

FIG. 5 is a more detailed illustration of the instrument head of FIG. 1, according to other various embodiments. As shown, the instrument head 108 includes, without limitation, a clamp 502, two or more electrodes 202, and a sheath 204. As previously discussed, the sheath 204 encloses wires that selectively couple the two or more electrodes 202 to external electrical components 106 (not shown). In various embodiments, the electrodes 202 are located at different positions along a length of the clamp. As shown, the clamp 502 includes a pair of jaws, each jaw including two or more teeth arranged along a longitudinal axis (e.g., a length axis) of the clamp 502. In various embodiments (without limitation), each electrode 202 can be coupled to one of the teeth of the clamp 502. The electrodes 202 are therefore located at different positions along the longitudinal axis. The jaws of the clamp 502 can be engaged (e.g., without limitation, by an electrical and/or mechanical actuator) to clamp a portion of tissue. The external electrical components 106 can selectively couple to respective pairs of electrodes (e.g., without limitation, a first electrode on an upper jaw at a selected position, and a second electrode on a lower jaw at the selected position). The external electrical components 106 can generate one or more impedance measurements of the tissue between the selected electrodes at the selected position of the clamp. Based on the impedance measurements for respective pairs of electrodes, the external electrical components 106 can generate a tissue type map of tissue types along the length of the clamp.

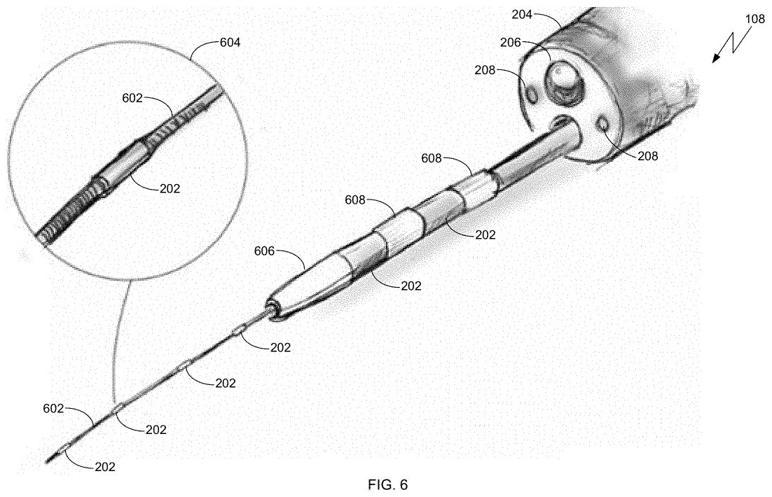

FIG. 6 is a more detailed illustration of the instrument head of FIG. 1, according to other various embodiments. As shown, the instrument head 108 includes, without limitation, a guidewire 602, an extension 606, two or more electrodes 202, a sheath 204, a camera 206, and two light sources 208. As previously discussed, the sheath 204 encloses wires that selectively couple the two or more electrodes 202 to external electrical components 106 (not shown).

As shown, the guidewire 602 extends in a forward direction from an extension 606 of the instrument head. The electrodes 202 are located at different positions along a length of the guidewire 602. As shown in the magnified view 604 of FIG. 6, each electrode 202 can enclose a circumference of the guidewire 602. As shown, some electrodes 202 are also located along a length of the extension 606. The extension 606 includes sections of electrically insulating material 608 that electrically insulate respective pairs of electrodes 202 located on the sheath 204. The external electrical components 106 can selectively couple to the electrodes 202 along the length of the extension 606 to record impedances and generate a first tissue type map. For example, (without limitation), the external electrical components 106 can generate a first tissue type map. Based on the first tissue type map, the external electrical components 106 can confirm that the instrument head 108 is correctly positioned at a targeted location 102, such as the location of a tumor. Then, the external electrical components 106 can record impedance measurements of the electrodes 202 located along the guidewire 602 to generate a second tissue type map of the tissue contacting the guidewire 602. The second tissue type map can confirm that the guidewire 602 is contacting tumor tissue type before or during operations associated with the guidewire 602 (e.g., without limitation, delivering a therapeutic agent or energy). In some embodiments, the external electrical components 106 perform operations associated with the guidewire based on the second tissue type map. For example (without limitation), the external electrical components 106 can activate the camera 206 or the light sources 208 when the second tissue type map indicates that the guidewire 602 is contacting a tumor tissue type.

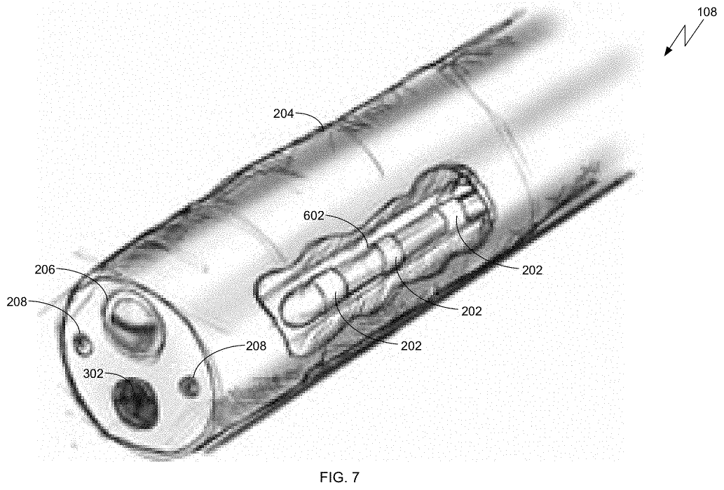

FIG. 7 is more detailed illustration of the instrument head of FIG. 1, according to other various embodiments. As shown, the instrument head 108 includes, without limitation, a sheath 204 including an aperture 302, a guidewire 602 including two or more electrodes 202, a sheath 204, a camera 206, and two light sources 208. As previously discussed, the sheath 204 encloses wires that selectively couple the two or more electrodes 202 to external electrical components 106 (not shown).

As shown, the guidewire 602 selectively retracts into the sheath 204. For example (without limitation), the guidewire 602 can fully retract into the sheath 204 while the instrument head 108 is being moved toward a targeted location 102. When the instrument head 108 is located at the targeted location 102, the guidewire 602 can extend through the aperture 302 of the sheath 204 to contact the tissue at the location 102. The external electrical components 106 can record impedance measurements of the electrodes 202 located along the extended guidewire 602 to generate a second tissue type map of the tissue contacting the guidewire 602. The second tissue type map can confirm that the guidewire 602 is contacting tumor tissue type before or during operations associated with the guidewire 602 (e.g., without limitation, delivering a therapeutic agent or energy). Also, after the medical procedure, the guidewire 602 can retract into the sheath 204 while the instrument head 108 is being removed from the location 102. The selective retraction and extension can protect the guidewire 602 from physical forces during movement of the instrument head 108.

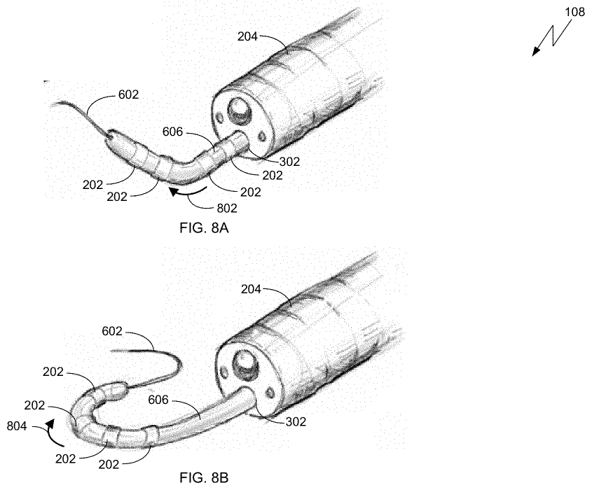

FIGS. 8A-8B are more detailed illustrations of the instrument head of FIG. 1, according to other various embodiments. As shown, the instrument head 108 includes, without limitation, a sheath 204 including an aperture 302, a guidewire 602, and an extension 606 including two or more electrodes 202. As previously discussed, the sheath 204 encloses wires that selectively couple the two or more electrodes 202 to external electrical components 106 (not shown).

As shown in FIG. 8A, the extension 606 extends from the aperture 302 of the sheath 204. The extension 606 bends at a bending location 802 along the length of the extension 606. In some embodiments, the extension 606 includes a shape memory material, such as Nitinol, which causes the extension 606 to bend at the bending location 802 when the extension 606 extends from the sheath 204. Alternatively or additionally, in some embodiments (not shown), the extension 606 includes a channel, and wires within the channel attach to one or more locations of an interior surface of the channel. Electrical and/or mechanical actuators can apply tension to one or more of the wires, causing the extension 606 to bend at the bending location 802. The bending of the extension 606 can position the guidewire 602 at a location of tissue that is difficult to reach with a straight extension 606.

As further shown in FIG. 8B, the extension 606 forms a curved shape 804 along a length of the extension 606. In some embodiments, the extension 606 includes a shape memory material, such as Nitinol, which causes the extension 606 to form the curved shape 804 when the extension 606 extends from the sheath 204. Alternatively or additionally, in some embodiments (not shown), the extension 606 includes a channel, and wires within the channel attach to one or more locations of an interior surface of the channel. Electrical and/or mechanical actuation of one or more of the wires can cause the extension 606 to form the curved shape 804. The curved shape 804 of the extension 606 can position the guidewire 602 at a location of tissue that is difficult to reach with a straight extension 606. In some embodiments, the extension 606 can selectively bend at a bending location 802 as shown in FIG. 8A (e.g., in response to actuation of one wire) and/or selectively form a curved shape 804 as shown in FIG. 8B (e.g., in response to actuation of two or more wires). Further, the external electrical components 106 can record impedance measurements of the one or more electrodes 202, or selected subsets thereof, to determine impedance measurements of tissue types at various locations near the instrument head 108. The external electrical components 106 can generate a tissue type map based on the impedance measurements.

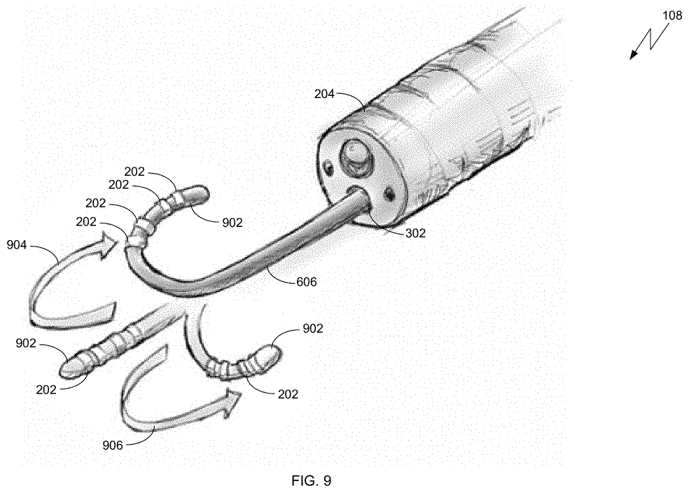

FIG. 9 is a more detailed illustration of the instrument head of FIG. 1, according to other various embodiments. As shown, the instrument head 108 includes, without limitation, a sheath 204 including an aperture 302 and an extension 606 including two or more electrodes 202. As previously discussed, the sheath 204 encloses wires that selectively couple the two or more electrodes 202 to external electrical components 106 (not shown).

As further shown in FIG. 9, the extension 606 selectively forms a first curved shape, a second curved shape, or a straight shape. For example (without limitation), when retracted into the sheath 204, the extension 606 can form a straight shape. When extended through the aperture 302 of the sheath 204, the extension 606 can form a straight shape, wherein a tip 902 of the extension 606 is oriented in a forward direction. The extension 606 can also selectively form a first curved shape 904 along a length of the extension 606 by curving in a first direction. The extension 606 can also selectively form a second curved shape 904 along a length of the extension 606 by curving in a second direction that is opposite the first direction. In some embodiments (not shown), the extension 606 includes a channel, and a set of wires within the channel attach to respective locations of an interior surface of the channel. Electrical and/or mechanical actuation of a first subset of the wires can cause the extension 606 to form the first curved shape 904. Electrical and/or mechanical actuation of a second subset of the wires can cause the extension 606 to form the second curved shape 906. Electrical and/or mechanical actuation of a third subset of the wires can cause the extension 606 to form the second curved shape 906. The selective shaping of the extension 606 between of the straight shape, the first curved shape 904, or the second curved shape 906 can position the tip 902 of the extension 606 at various locations of tissue that are difficult to reach with a straight extension 606. Further, the external electrical components 106 can record impedance measurements of the one or more electrodes 202, or selected subsets thereof, to determine impedance measurements of tissue types at various locations near the instrument head 108. The external electrical components 106 can generate a tissue type map based on the impedance measurements.

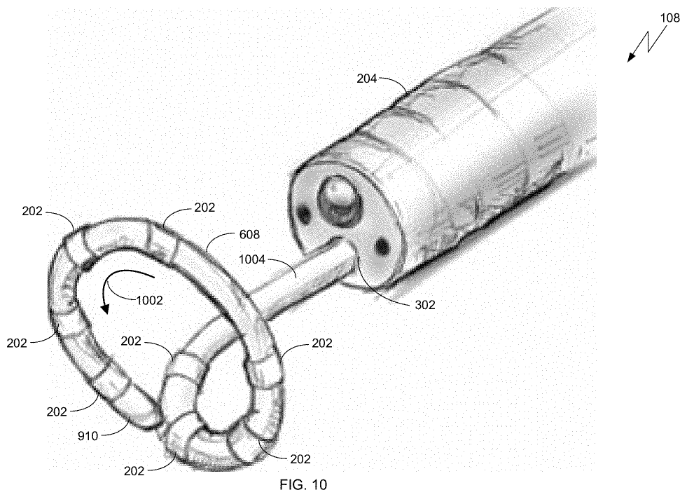

FIG. 10 is a more detailed illustration of the instrument head of FIG. 1, according to other various embodiments. As shown, the instrument head 108 includes, without limitation, a sheath 204 including an aperture 302 and an extension 606 including two or more electrodes 202. As previously discussed, the sheath 204 encloses wires that selectively couple the two or more electrodes 202 to external electrical components 106 (not shown).

As further shown in FIG. 10, the extension 606 selectively forms a circular shape 1002 that encircles a longitudinal axis (e.g., a length axis) of the instrument head. For example (without limitation), when retracted into the sheath 204, the extension 606 can form a straight shape. When extended through the aperture 302 of the sheath 204, a first portion 1004 of the extension 606 can extend in a straight or forward direction. Another portion of the extension 606 that is distal to the first portion 1004 can form a circular shape 1002. For example (without limitation), the extension 606 can include a shape memory material, such as Nitinol, which forms the circular shape 1002 in an unconstrained state. Alternatively or additionally, in some embodiments, the extension 606 includes a channel (not shown), and a set of wires within the channel attach to respective locations of an interior surface of the channel. Electrical and/or mechanical actuation of a first subset of the wires can selectively cause the extension 606 to form the circular shape 1002. The external electrical components 106 can record impedance measurements of the one or more electrodes 202, or selected subsets thereof, to determine impedance measurements of tissue types at various locations along the circular shape 1002. The external electrical components 106 can generate a circularly shaped tissue type map based on the impedance measurements.

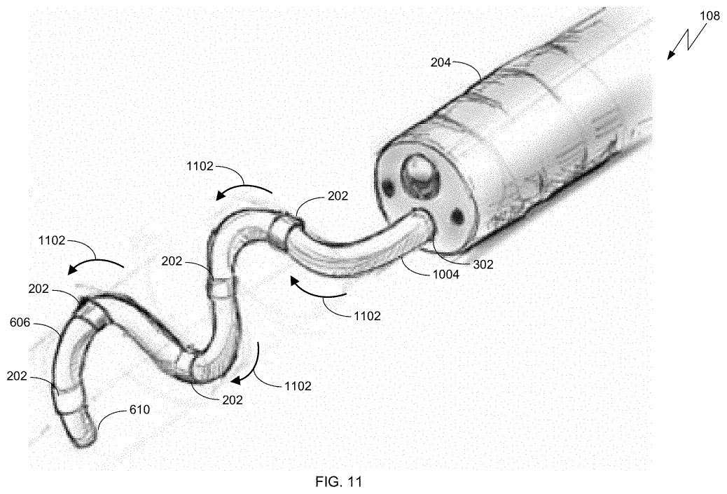

FIG. 11 is a more detailed illustration of the instrument head of FIG. 1, according to other various embodiments. As shown, the instrument head 108 includes, without limitation, a sheath 204 including an aperture 302 and an extension 606 including two or more electrodes 202. As previously discussed, the sheath 204 encloses wires that selectively couple the two or more electrodes 202 to external electrical components 106 (not shown).

As further shown in FIG. 11, the extension 606 selectively forms a wave shape 1102 relative to a longitudinal axis (e.g., a length axis) of the instrument head. For example (without limitation), when retracted into the sheath 204, the extension 606 can form a straight shape. When extended through the aperture 302 of the sheath 204, a first portion 1004 of the extension 606 can extend in a straight direction. Another portion of the extension 606 that is distal to the first portion 1004 can form a wave shape 1102. For example (without limitation), the extension 606 can include a shape memory material, such as Nitinol, which forms the wave shape 1102 in an unconstrained state. Alternatively or additionally, in some embodiments, the extension 606 includes a channel (not shown), and a set of wires within the channel attach to respective locations of an interior surface of the channel. Electrical and/or mechanical actuation of the wires can selectively cause the extension 606 to form the wave shape 1102. The external electrical components 106 can record impedance measurements of the one or more electrodes 202, or selected subsets thereof, to determine impedance measurements of tissue types at various locations along the wave shape 1102. The external electrical components 106 can generate a wave-shaped tissue type map based on the impedance measurements.

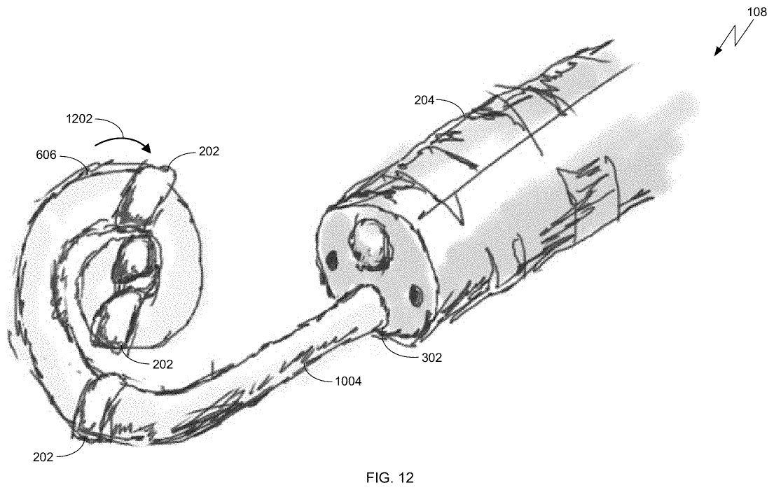

FIG. 12 is a more detailed illustration of the instrument head of FIG. 1, according to other various embodiments. As shown, the instrument head 108 includes, without limitation, a sheath 204 including an aperture 302 and an extension 606 including two or more electrodes 202. As previously discussed, the sheath 204 encloses wires that selectively couple the two or more electrodes 202 to external electrical components 106 (not shown).

As further shown in FIG. 12, the extension 606 selectively forms a spiral shape 1202. For example (without limitation), when retracted into the sheath 204, the extension 606 can form a straight shape. When extended through the aperture 302 of the sheath 204, a first portion 1004 of the extension 606 can extend in a straight direction. Another portion of the extension 606 that is distal to the first portion 1004 can form a spiral shape 1202. For example (without limitation), the extension 606 can include a shape memory material, such as Nitinol, which forms the spiral shape 1202 in an unconstrained state. Alternatively or additionally, in some embodiments, the extension 606 includes a channel (not shown), and a set of wires within the channel attach to respective locations of an interior surface of the channel. Electrical and/or mechanical actuation of the wires can selectively cause the extension 606 to form the spiral shape 1202. The external electrical components 106 can record impedance measurements of the one or more electrodes 202, or selected subsets thereof, to determine impedance measurements of tissue types at various locations along the spiral shape 1202. The external electrical components 106 can generate a spiral-shaped tissue type map based on the impedance measurements. In various embodiments, a plane of the spiral shape 1202 can be oriented in a parallel orientation, a perpendicular orientation, and/or an oblique orientation relative to a longitudinal axis (e.g., a length axis) of the instrument head 108.

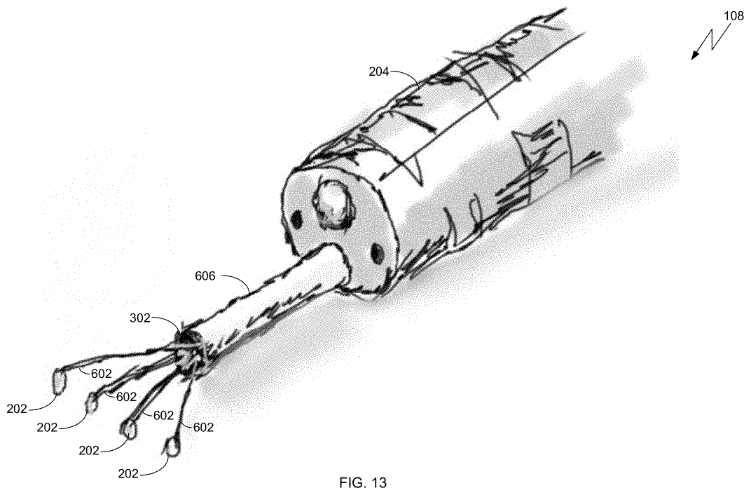

FIG. 13 is a more detailed illustration of the instrument head of FIG. 1, according to other various embodiments. As shown, the instrument head 108 includes, without limitation, a sheath 204, an extension 606 including an aperture 302, and at least two guidewires that respectively include an electrode 202. As previously discussed, the sheath 204 encloses wires that selectively couple the electrodes 202 to external electrical components 106 (not shown).

As shown, the at least two guidewires 602 extend from the extension 606 in a rake configuration. As shown, each of the at least two guidewires 602 extends from the instrument head 108 in a different direction. More particularly, each of the two or more guidewire 602 extends from the aperture 302 of the extension 606 in a different direction. Each of the two or more electrodes 202 is located at a tip of one of the guidewires 602. The rake configuration of the guidewires 602 spreads the electrodes 202 in a lateral direction relative to a longitudinal axis (e.g., a length axis) of the instrument head 108. of the extension 606 that is distal to the first portion 1004 can form a spiral shape 1202. For example (without limitation), each guidewire 602 can include a shape memory material, such as Nitinol. Each guidewire can include a bending location that bends the guidewire 602 in a particular direction. When retracted into the extension 606, each guidewire 602 can form a straight shape. When extended through the aperture 302 of the extension 606, each guidewire 602 can form a bent shape in which the guidewire 602 bends in a direction relative to the longitudinal axis of the instrument head 108. The external electrical components 106 can record impedance measurements of the two or more electrodes 202, including selected subsets thereof, to determine impedance measurements of tissue types between various pairs of guidewires 602 of the rake. The external electrical components 106 can generate a linear tissue type map based on the impedance measurements, wherein the linear tissue type map extends in a lateral direction relative to the longitudinal axis of the instrument head 108. In various embodiments, the lateral direction can be oriented perpendicular to the longitudinal axis of the instrument head 108 (as shown), parallel to the longitudinal axis of the instrument head, or in an oblique direction relative to the longitudinal axis of the instrument head.

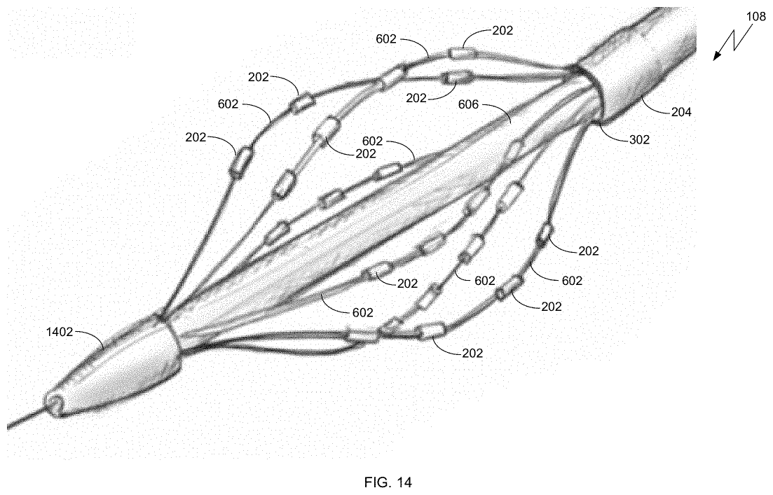

FIG. 14 is a more detailed illustration of the instrument head of FIG. 1, according to other various embodiments. As shown, the instrument head 108 includes, without limitation, a sheath 204 including an aperture 302, an extension 606 terminating in an extension tip 1402, and two or more guidewires 602, each extending between the sheath and the extension tip 1402, wherein each guidewire 602 includes one or more electrodes 202 respectively located at a lateral position along the guidewire 602. As previously discussed, the sheath 204 encloses wires that selectively couple the electrodes 202 to external electrical components 106 (not shown).

As shown, the guidewires 602 extend laterally from the sheath 204 to the extension tip 1402. Each guidewire 602 protrudes in an outward direction relative to a longitudinal axis of the extension 606. Further, each guidewire 602 protrudes in different outward direction relative to the longitudinal axis of the extension 606 than the other guidewires 602. For example (without limitation), each guidewire 602 can include a shape memory material, such as Nitinol. When retracted into the sheath 204, each guidewire 602 can form a straight shape. In an unconstrained state, each guidewire can form a protruding shape that protrudes the guidewire 602 in a particular direction. When extended through the aperture 302 of the sheath 204, each guidewire 602 can form a protruding shape in which the guidewire 602 protrudes in an outward direction relative to the longitudinal axis of the extension 606. The external electrical components 106 can record impedance measurements of two or more electrodes 202, including selected subsets thereof, to determine impedance measurements and tissue types of the tissue near the instrument head 108. For example (without limitation), the external electrical components 106 can record a first set of impedance measurements between two or more electrodes 202 at a first position along the lengths of the respective guidewires 602 and a second set of impedance measurements between two or more electrodes 202 at a second position along the lengths of the respective guidewires 602. As another example (without limitation), the external electrical components 106 can record a first set of impedance measurements between two or more electrodes 202 of a first guidewires 602 and a second set of impedance measurements between two or more electrodes 202 of a second guidewire 602. The external electrical components 106 can generate a volumetric tissue type map based on the impedance measurements, wherein the volumetric tissue type map includes layers of tissue types in various directions, distances, and lateral positions relative to the extension 606.

FIGS. 15A-B are more detailed illustrations of the instrument head of FIG. 1, according to other various embodiments. As shown, the instrument head 108 includes, without limitation, a sheath 204 including an aperture 302, an extension 606 terminating in an extension tip 1402, a wire 1502 that extends along the extension 606 through the extension tip 1402, and two or more guidewires 602, each extending between the sheath and the extension tip 1402, wherein each guidewire 602 includes one or more electrodes 202 respectively located at a lateral position along the guidewire 602. As previously discussed, the sheath 204 encloses wires that selectively couple the electrodes 202 to external electrical components 106 (not shown).

As shown in FIG. 15A, the extension tip 1402 is positioned at a first position along the wire 1502. As a result, the guidewires 602, which extend laterally from the sheath 204 to the extension tip 1402, are flush and/or parallel with the extension 606. The external electrical components 106 can record a first set impedance measurements of two or more electrodes 202 in the flush and/or parallel orientation, including selected subsets thereof, to determine impedance measurements and tissue types of tissue near the instrument head 108. For example (without limitation), the external electrical components 106 can record a first subset of impedance measurements between two or more electrodes 202 at a first position along the lengths of the respective guidewires 602 and a second subset of impedance measurements between two or more electrodes 202 at a second position along the lengths of the respective guidewires 602. As another example (without limitation), the external electrical components 106 can record a first subset of impedance measurements between two or more electrodes 202 of a first guidewires 602 and a second subset of impedance measurements between two or more electrodes 202 of a second guidewire 602.

As shown in FIG. 15B, the extension tip 1402 retracts (e.g., moves in a retraction direction 1504) relative to the extension 606. As a result, the extension tip 1402 in a retraction direction 1504 compresses each guidewire 602, causing each guidewire 602 to protrude in an outward direction 1506 relative to a longitudinal axis (e.g., a length axis) of the extension 606. In some embodiments (not shown), a wire connected to the extension tip 1402 can be electrically and/or mechanically actuated to create tension that pulls the extension tip 1402 in the retraction direction 1504. Due to the coupling of the guidewires 602 and the extension tip 1402, retracting the extension tip 1402 changes a shape of the guidewires 602 from the flush or parallel configuration shown in FIG. 15A to the protruding configuration shown in FIG. 15B. Further, due to the arrangement of the guidewires 602 around the extension 606, each guidewire 602 protrudes in different outward direction relative to the longitudinal axis of the extension 606 than the other guidewires 602. The external electrical components 106 can record a second set of impedance measurements of two or more electrodes 202, including selected subsets thereof, to determine impedance measurements of tissue types between various pairs of guidewires 602 of the protruding shapes. For example (without limitation), the external electrical components 106 can record a first subset of impedance measurements between two or more electrodes 202 at a first position along the lengths of the respective guidewires 602 and a second subset of impedance measurements between two or more electrodes 202 at a second position along the lengths of the respective guidewires 602. As another example (without limitation), the external electrical components 106 can record a first subset of impedance measurements between two or more electrodes 202 of a first guidewires 602 and a second subset of impedance measurements between two or more electrodes 202 of a second guidewire 602. Based on the first set of impedance measurements and the second set of impedance measurements, the external electrical components 106 can generate a volumetric tissue type map, wherein the volumetric tissue type map includes layers of tissue types in various directions, distances, and lateral positions relative to the extension 606.

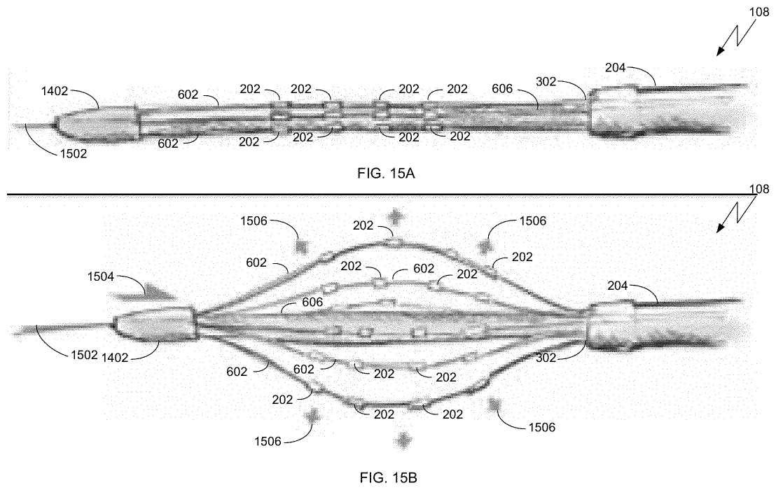

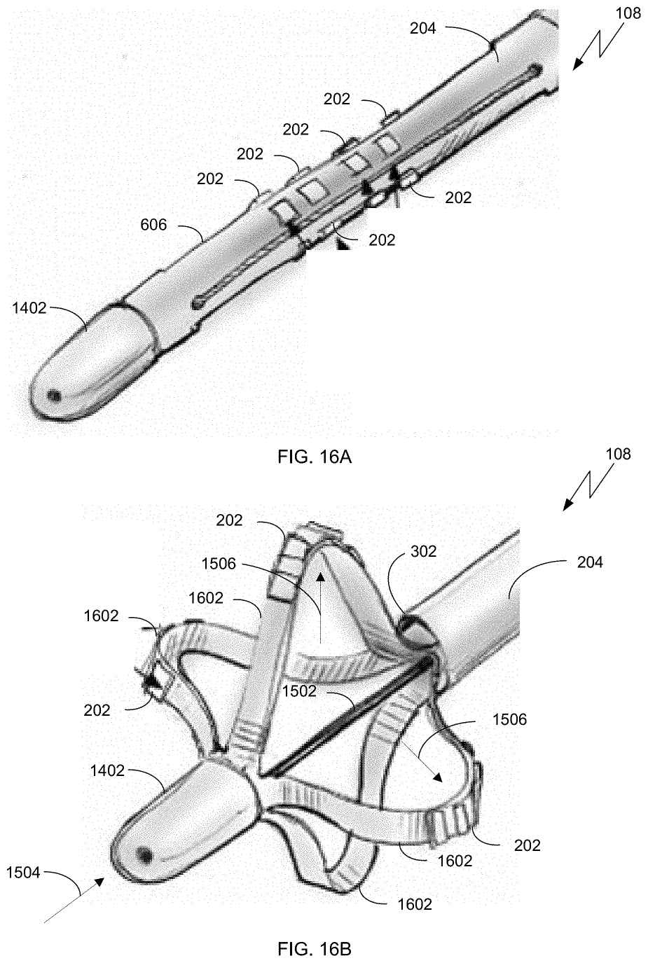

FIGS. 16A-B are more detailed illustrations of the instrument head of FIG. 1, according to other various embodiments. As shown, the instrument head 108 includes, without limitation, a sheath 204 including an aperture 302, an extension tip 1402, a wire 1502 that extends along the extension 606 to the extension tip 1402, and two or more bands 1602, each band 1602 extending between the sheath and the extension tip 1402, wherein each band 1602 includes one or more electrodes 202 respectively located at a lateral position along the band 1602. As previously discussed, the sheath 204 encloses wires that selectively couple the electrodes 202 to external electrical components 106 (not shown).

As shown in FIG. 16A, the extension tip 1402 is positioned at a first position along the wire 1502. As a result, the bands 1602, which extend laterally from the sheath 204 to the extension tip 1402, are parallel with the wire 1502. The external electrical components 106 can record a first set impedance measurements of two or more electrodes 202 in the parallel orientation, including selected subsets thereof, to determine impedance measurements and tissue types of tissue near the instrument head 108. For example (without limitation), the external electrical components 106 can record a first subset of impedance measurements between two or more electrodes 202 at a first position along the lengths of the respective bands 1602 and a second subset of impedance measurements between two or more electrodes 202 at a second position along the lengths of the respective bands 1602. As another example (without limitation), the external electrical components 106 can record a first subset of impedance measurements between two or more electrodes 202 of a first band 1602 and a second subset of impedance measurements between two or more electrodes 202 of a second band 1602.

As shown in FIG. 16B, the extension tip 1402 retracts (e.g., moves in a retraction direction 1504). As a result, the extension tip 1402 compresses each band 1602, causing each band 1602 to protrude in an outward direction 1506 relative to a longitudinal axis (e.g., a length axis) of the wire 1502. In some embodiments, the wire 1502 can be electrically and/or mechanically actuated to create tension that pulls the extension tip 1402 in the retraction direction 1504. Due to the coupling of the bands 1602 and the extension tip 1402, retracting the extension tip 1402 changes a shape of the bands 1602 from the parallel configuration shown in FIG. 16A to the protruding configuration shown in FIG. 16B. Further, due to the arrangement of the bands 1602 around the wire 1502, each band 1602 protrudes in different outward direction relative to the longitudinal axis of the wire 1502 than the other bands 1602. The external electrical components 106 can record a second set of impedance measurements of two or more electrodes 202, including selected subsets thereof, to determine impedance measurements of tissue types between various pairs of bands 1602. For example (without limitation), the external electrical components 106 can record a first subset of impedance measurements between two or more electrodes 202 at a first position along the lengths of the respective bands 1602 and a second subset of impedance measurements between two or more electrodes 202 at a second position along the lengths of the respective bands 1602. As another example (without limitation), the external electrical components 106 can record a first subset of impedance measurements between two or more electrodes 202 of a first band 1602 and a second subset of impedance measurements between two or more electrodes 202 of a second band 1602. Based on the first set of impedance measurements and the second set of impedance measurements, the external electrical components 106 can generate a volumetric tissue type map, wherein the volumetric tissue type map includes layers of tissue types in various directions, distances, and lateral positions relative to the wire 1502.

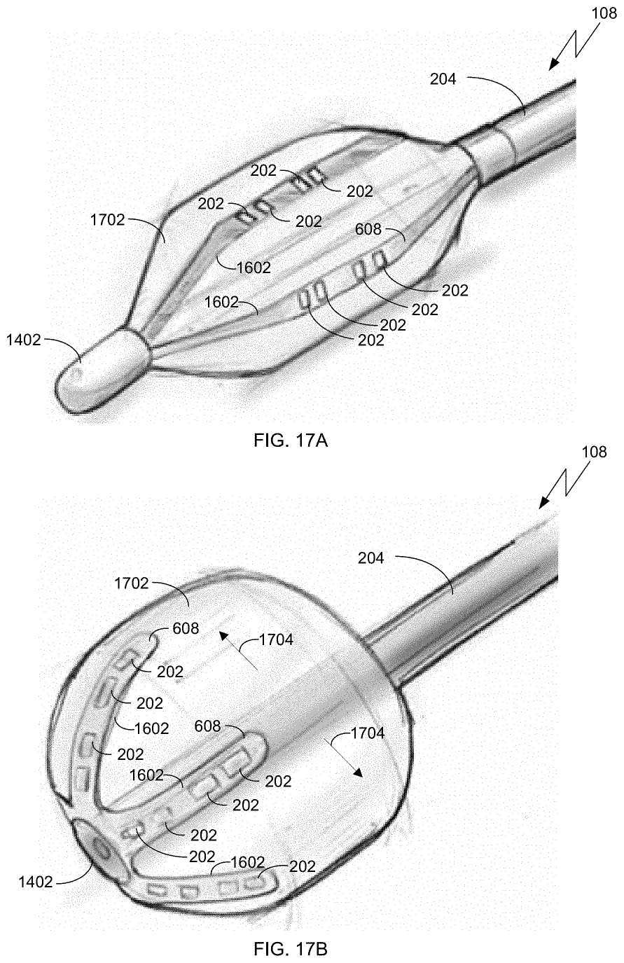

FIGS. 17A-B are more detailed illustrations of the instrument head of FIG. 1, according to other various embodiments. As shown, the instrument head 108 includes, without limitation, a sheath 204, an extension tip 1402, and a balloon 1702 including two or more bands 1602, each band 1602 extending from the extension tip 1402 along a surface of the balloon 1702, wherein each band 1602 includes one or more electrodes 202 respectively located at a lateral position along the band 1602. As previously discussed, the sheath 204 encloses wires that selectively couple the electrodes 202 to external electrical components 106 (not shown).

As shown in FIG. 17A, the balloon 1702 is in a collapsed configuration. As a result, the bands 1602, each extending laterally from the extension tip 1402 along the surface of the balloon 1702 in a different direction, are parallel with a longitudinal axis (e.g., a length axis) of the instrument head 108. The external electrical components 106 can record a first set impedance measurements of two or more electrodes 202 in the parallel orientation, including selected subsets thereof, to determine impedance measurements and tissue types of tissue near the instrument head 108. For example (without limitation), the external electrical components 106 can record a first subset of impedance measurements between two or more electrodes 202 at a first position along the lengths of the respective bands 1602 and a second subset of impedance measurements between two or more electrodes 202 at a second position along the lengths of the respective bands 1602. As another example (without limitation), the external electrical components 106 can record a first subset of impedance measurements between two or more electrodes 202 of a first band 1602 and a second subset of impedance measurements between two or more electrodes 202 of a second band 1602.

As shown in FIG. 17B, the balloon 1702 is in an expanded configuration in which the surface of the balloon 1702 expands in an outward direction 1704 relative to the longitudinal axis of the instrument head 108. As a result, each band 1602 located on the surface of the balloon 1702 protrudes in an outward direction 1506 relative to the longitudinal axis of the instrument head 108. In some embodiments, the balloon 1702 can be electrically and/or mechanically inflated with air or any other medium. Due to the location of the bands 1602 on the surface of the balloon 1702, inflating the balloon 1702 changes a shape of the bands 1602 from the parallel configuration shown in FIG. 17A to the protruding configuration shown in FIG. 17B. Further, due to the arrangement of the bands 1602 around the longitudinal axis of the instrument head 108, each band 1602 protrudes in different outward directions 1704 relative to the longitudinal axis of the instrument head 108 than the other bands 1602. The external electrical components 106 can record a second set of impedance measurements of two or more electrodes 202, including selected subsets thereof, to determine impedance measurements of tissue types between various pairs of bands 1602. For example (without limitation), the external electrical components 106 can record a first subset of impedance measurements between two or more electrodes 202 at a first position along the lengths of the respective bands 1602 and a second subset of impedance measurements between two or more electrodes 202 at a second position along the lengths of the respective bands 1602. As another example (without limitation), the external electrical components 106 can record a first subset of impedance measurements between two or more electrodes 202 of a first band 1602 and a second subset of impedance measurements between two or more electrodes 202 of a second band 1602. Based on the first set of impedance measurements and the second set of impedance measurements, the external electrical components 106 can generate a volumetric tissue type map, wherein the volumetric tissue type map includes layers of tissue types in various directions, distances, and lateral positions relative to the longitudinal axis of the instrument head 108.

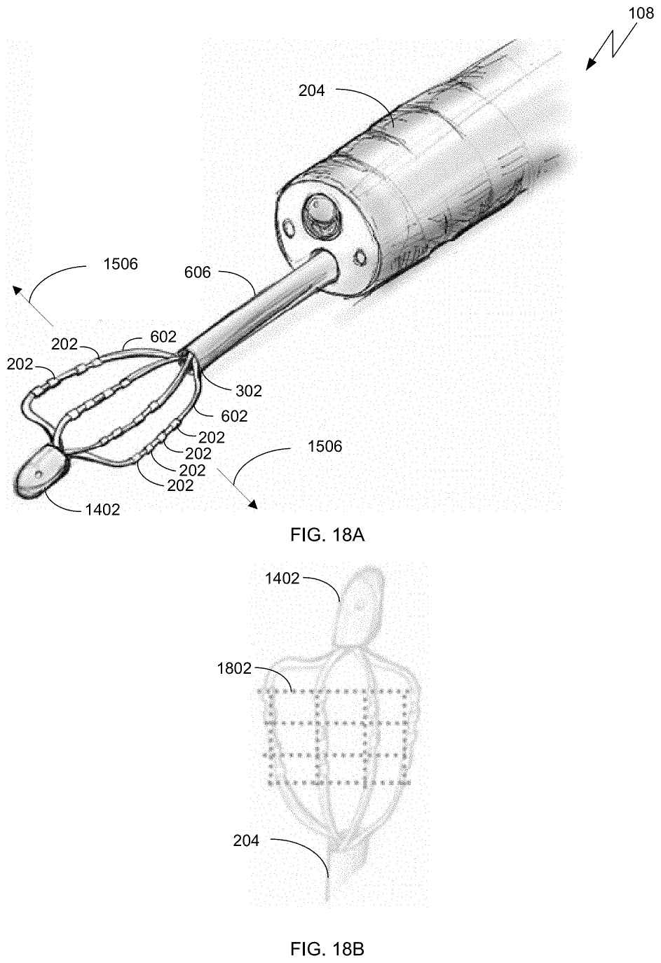

FIG. 18A is a more detailed illustration of the instrument head of FIG. 1, according to other various embodiments. As shown, the instrument head 108 includes, without limitation, a sheath 204, an extension 606 including an aperture 302 and an extension tip 1402, a wire 1502 that extends along the extension 606 through the extension tip 1402, and two or more guidewires 602, each guidewire 602 extending between the extension and the extension tip 1402, wherein each guidewire 602 includes one or more electrodes 202 respectively located at a lateral position along the guidewire 602. As previously discussed, the sheath 204 encloses wires that selectively couple the electrodes 202 to external electrical components 106 (not shown).

As shown in FIG. 18A, each guidewires 602 extend laterally from the sheath 204 to the extension tip 1402. Also, each guidewire 602 protrudes in an outward direction 1506 relative to a longitudinal axis (e.g., a length axis) of the extension 606. For example (without limitation), the guidewires 602 can retract into the sheath 204 and can be in a parallel configuration when retracted into the sheath 204. When extended from the sheath 204, each guidewire 602 can extend in a different direction relative to the other guidewires 602. Due to the arrangement of the guidewires 602 around the extension 606, each guidewire 602 protrudes in different outward direction relative to the longitudinal axis of the extension 606 than the other guidewires 602. The external electrical components 106 can record impedance measurements of two or more electrodes 202, including selected subsets thereof, to determine impedance measurements of tissue types between various pairs of guidewires 602 of the protruding shapes. For example (without limitation), the external electrical components 106 can record a first set of impedance measurements between two or more electrodes 202 at a first position along the lengths of the respective guidewires 602 and a second set of impedance measurements between two or more electrodes 202 at a second position along the lengths of the respective guidewires 602. As another example (without limitation), the external electrical components 106 can record a first set of impedance measurements between two or more electrodes 202 of a first guidewires 602 and a second set of impedance measurements between two or more electrodes 202 of a second guidewire 602.

FIG. 18B is an illustration of a grid pattern 1802 associated with the instrument head of FIG. 18A. As shown, each electrode 202 is located at a node of the grid pattern 1802 at a particular position along a longitudinal axis (e.g., a length axis) of the extension 606 and/or a different lateral distance from the extension 606. Based on impedance measurements recorded between various pairs of nodes of the graph pattern 180, the external electrical components 106 can generate a grid tissue type map, wherein the grid tissue type map includes layers of tissue types in various directions, distances, and lateral positions relative to the extension 606.

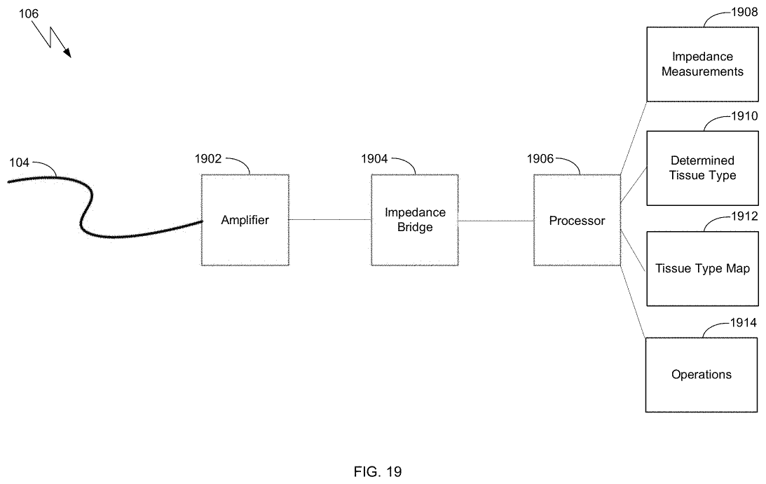

FIG. 19 is a more detailed illustration of the external electrical components of FIG. 1, according to various embodiments. As shown, the external electrical components 106 include wires 104, an amplifier 1902, an impedance bridge 1904, and a processor 1906. The wires 104 conduct current at various frequencies between a selected two or more electrodes 202 and the external electrical components 106. In various embodiments, the amplifier 1902 is an analog interface amplifier that amplifies a supplied voltage and/or a return voltage while the wires 104 conduct current at various frequencies between the impedance bridge 1904 and the selected two or more electrodes 202. In various embodiments, the impedance bridge 1904 is an impedance load that the processor 1906 measures to determine an impedance of a circuit including the impedance bridge 1904, the amplifier 1902, and the selected two or more electrodes 202. The processor 1906 generates frequencies for a current that the wires 104 conduct between the impedance bridge 1904 and the selected two or more electrodes 202.

While the wires 104 conduct current at various frequencies, the processor 1906 records one or more impedance measurements 1908 of the circuit including the at selected least two electrodes 202. The processor 1906 determines, based on the impedance measurements 1908, a tissue type 1910 of a portion of tissue between the selected two or more electrodes 202. In various embodiments, the processor 1906 determines the tissue type 1910 by comparing the impedance measurements 1908 with one or more characteristic impedance measurements associated with one or more tissue types. For example and without limitation, based on the comparing, the processor 1906 can determine which tissue type is associated with characteristic impedance measurements that are closest to the impedance measurements of the portion of tissue between the selected two or more electrodes 202. In various embodiments, the processor 1906 can determine a Cole relaxation frequency of the portion of tissue based on the impedance measurements 1908, and can compare the Cole relaxation frequency to one or more characteristic Cole relaxation frequencies of one or more tissue types. The Cole relaxation frequency corresponds to a frequency associated with a greatest impedance measurement 1908 included in the one or more impedance measurements 1908. In various embodiments, the Cole relaxation frequency is a frequency of a maximum normalized impedance measurement of the portion of tissue between the two or more electrodes 202. For example and without limitation, based on a Cole relaxation frequency below a threshold frequency (e.g., 10⁵ Hz), the processor 1906 can determine that the portion of tissue between the selected two or more electrodes 202 is a non-tumor tissue type. Similarly, for example and without limitation, based on a Cole relaxation frequency above the threshold frequency, the processor 1906 can determine that the portion of tissue between the two or more electrodes 202 is a tumor tissue type.

In various embodiments, the processor 1906 determines a tissue type map 1912 based on the determined tissue types 1910. For example (without limitation), the processor 1906 selectively delivers current to a sequence of selected two or more electrodes 202 that are positioned at respective locations relative to the instrument head 108 (e.g., a first pair of electrodes on a left side of the instrument head 108 and a second pair of electrodes on a right side of the instrument head 108). Alternatively or additionally, as another example (without limitation), the processor 1906 selectively delivers current to the two or more electrodes 202 taken at a first point in time when the instrument head 108 is positioned at a first location within a patient's body and a second point in time when the instrument head 108 is positioned at a second location within the patient's body. Based on the determined tissue types 1910 of the sets of electrodes positioned at respective locations and/or at different points in time, the processor 1906 determines a tissue type map 1912 of tissue types 1910 near the instrument head 108. For example (without limitation), based on the grid pattern 1802 of FIG. 18B, the processor 1906 can determine the tissue types 1910 between respective pairs of electrodes 202 positioned at adjacent nodes of the grid pattern 1802. The tissue type map 1912 indicates the determined tissue types 1910 of the tissue between each pair of adjacent nodes of the grid pattern 1802.

In various embodiments, the processor 1906 performs one or more operations 1914 to control a medical device tool based on the determined tissue type 610. For example and without limitation, in various embodiments in which the instrument head 108 includes a camera 206, the processor 1906 can perform operations 1914 that include activating the camera 206 to capture an image of the tissue at the location 102 of the instrument head 108. For example and without limitation, in various embodiments in which the instrument head 108 includes a light source 208, the processor 1906 can perform operations 1914 that include activating the light source 208 to illuminate the tissue at the location 102 of the instrument head 108. For example and without limitation, in various embodiments in which the instrument head 108 includes a therapeutic drug delivery tool, the processor 1906 can perform operations 1914 that include activating the therapeutic drug delivery tool to deliver one or more therapeutic drugs to the tissue at the location 102 of the instrument head 108. For example and without limitation, in various embodiments in which the instrument head 108 includes an energy delivery tool, the processor 1806 can perform operations 1914 that include activating the energy delivery tool to deliver energy to the tissue at the location 102 of the instrument head 108. For example and without limitation, in various embodiments in which the instrument head 108 includes a tissue sample extraction tool, the processor 1906 can perform operations 1914 that include activating the tissue sample extraction tool to extract a tissue sample from the tissue at the location 102 of the instrument head 108.

In various embodiments, the processor 1906 presents the tissue type map 1912 indicating the determined tissue types 1910 at the location 102 of the instrument head 108. For example and without limitation, the processor 1906 can display the tissue type map 1912 using a visual output (e.g., a light-emitting diode, a liquid crystal display, or the like). For example and without limitation, where the target location 102 is a tumor, the displayed tissue type map 1912 can indicate that a determined tumor tissue type at a particular position relative to the instrument head 108 (e.g., on a left side or a right side of the instrument head 108) matches an expected tissue type of the tissue at the location 102. Presenting the indication can inform a user of the medical device 100 that the location 102 of the instrument head 108 matches a target location. Further, in various embodiments, the processor 1906 performs the one or more operations 1914 to control a medical device tool based on presenting the tissue type map 1912 and receiving a signal to activate the medical device tool.

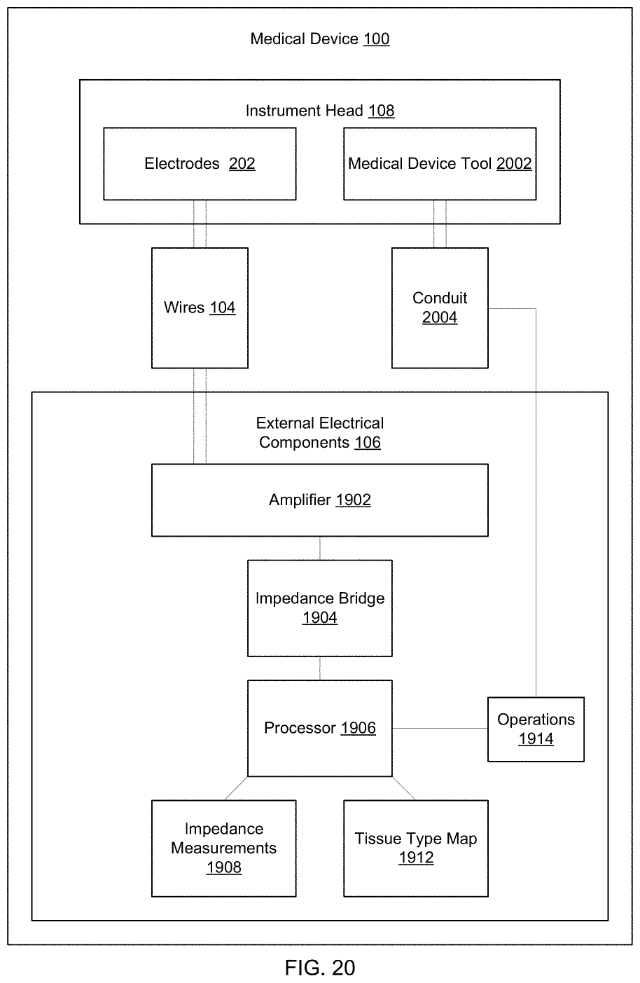

FIG. 20 is a more detailed illustration of the medical device 100 of FIG. 1, according to various embodiments. As shown, the medical device 100 includes an instrument head 108 and external electrical components 106. As shown, the instrument head 108 includes two or more electrodes 202 that are selectively coupled to the external electrical components 106 by wires 104. In various embodiments, without limitation, each of the two or more electrodes 202 is coupled to the external electrical components 106 by one wire 104 or by respective wires of a plurality of wires 104. As shown, the instrument head 108 also includes a medical device tool 2002, such as and without limitation, a therapeutic drug delivery tool, an energy delivery tool, or a tissue sample extraction tool. In various embodiments, the instrument head 108 includes, without limitation, two or more medical device tools 2002, which can be of one kind or of different kinds.

As shown, the external electrical components 106 include an amplifier 1902, an impedance bridge 1904, and a processor 1906. The amplifier 1902 amplifies a supplied voltage and/or a return voltage while the wires 104 conduct current at various frequencies between the impedance bridge 1904 and a selected set of electrodes of the two or more electrodes 202. The impedance bridge 1904 is an impedance load that the processor 1906 measures to determine an impedance of a circuit including the impedance bridge 1904, the amplifier 1902, the wires 104, and the two or more electrodes 202. The processor 1906 records, at various frequencies, one or more impedance measurements 1908. The processor 1906 determines a tissue type map 1912 of tissue types at the location 102 of the instrument head 108 based on the impedance measurements 1908. In various embodiments and without limitation, the processor 1906 determines the tissue types indicated by the respective impedance measurements 1908 based on a Cole relaxation frequency of the portion of tissue contacting the selected two or more electrodes 202. In various embodiments and without limitation, the processor 1906 determines the tissue type map 1912 as areas of tumor tissue types and/or non-tumor tissue types. In various embodiments and without limitation, based on the tissue type map 1912, the processor 1906 determines that tissue types 1910 at the location 102 of the instrument head 108 match the expected tissue types of tissue at a target location, which indicates or confirms that the instrument head 108 is positioned at the target location 102. For example and without limitation, if the target location 102 is a tumor, the processor 1906 can determine whether the instrument head 108 is positioned at a target location 102 by determining that the tissue types 1918 indicated by the tissue type map 1912 are a tumor tissue type.

As shown, the processor 1906 is coupled to a conduit 2004 of the medical device tool 2002. Based on the tissue type map 1912, the processor 1906 performs one or more operations 1914 to control the medical device tool 2002. In various embodiments and without limitation, the medical device tool 2002 includes a therapeutic drug delivery tool, and the processor 1906 performs an operation 1914 of causing the medical device tool 2002 to deliver one or more therapeutic drugs to tissue at the location 102 of the instrument head 108. For example and without limitation, the processor 1906 can cause one or more therapeutic drugs through one or more drug delivery conduits to and through the therapeutic drug delivery tool. In various embodiments and without limitation, the medical device tool 2002 includes an energy delivery tool, and the processor 1906 performs an operation 1914 of causing the conduit 2004 and the medical device tool 2002 to deliver energy to tissue at the location 102 of the instrument head 108. For example and without limitation, the processor 1906 can current to be conducted through wires in the conduit 2004 to and through the energy delivery tool. In various embodiments and without limitation, the medical device tool 2002 is a tissue sample extraction tool, and the processor 2006 performs an operation 1914 of causing the tissue sample extraction tool to extract a tissue sample from tissue at the location 102 of the instrument head 108. For example and without limitation, the external electrical components 106 can include an actuator coupled to the tissue sample extraction tool by wires in the conduit 2004, and the processor 1906 can activate the actuator to cause the tissue sample extraction tool to extract the tissue sample.

In various embodiments, the medical device 100 reports the tissue type map 1912 to a user of the medical device 100. For example and without limitation, the medical device 100 can display the tissue type map 1912 using a visual output (e.g., a liquid crystal display (LCD), a light-emitting diode (LED) display to present a visual indication of determined tissue types 1910, such as a light, symbol, text, graphic, or the like). In various embodiments and without limitation, the processor 1906 can include, in the displayed tissue type map 1912, an indication that the determined tissue types 1910 match the expected tissue type of tissue at a target location (e.g., using a visual output, an audio output, or the like).

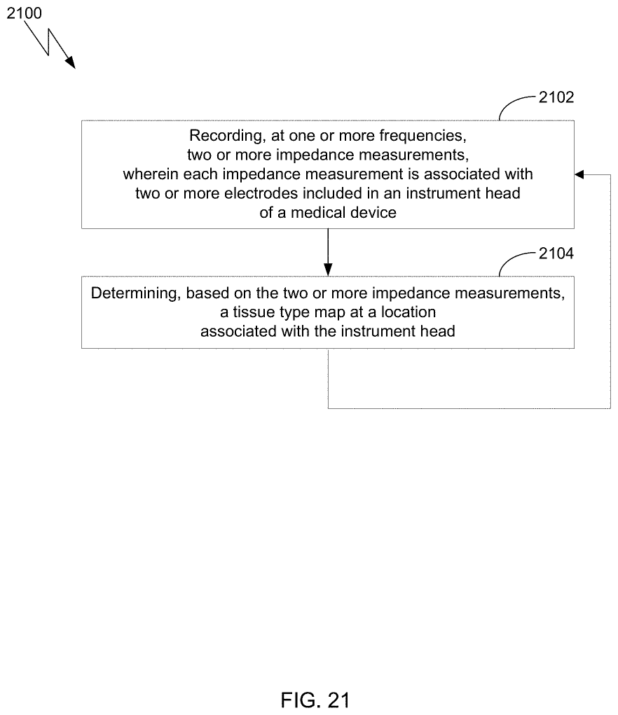

FIG. 21 is a flow diagram of method steps for controlling the medical device 100 of FIG. 1, according to various embodiments. Although the method steps are described in conjunction with the systems of FIGS. 1-20, persons skilled in the art will understand that any system configured to perform the method steps, in any order, falls within the scope of the present invention.

As shown, a method 2100 begins at step 2102, where a processor 1906 records, at one or more frequencies, two or more impedance measurements 1908, wherein each impedance measurement 1908 is associated with two or more electrodes 202 included in an instrument head 108 of the medical device 100. In various embodiments and without limitation, the processor 1906 determines a Cole relaxation frequency of tissue between the selected two or more electrodes 202 in the instrument head 108, e.g., as a frequency of a maximum normalized impedance measurement of the tissue between the selected two or more electrodes 202.

At step 2104, the processor 1906 determines, based on the two or more impedance measurements 1908, a tissue type map 1912 at a location associated with the instrument head 108. In various embodiments and without limitation, the processor 1906 determines the tissue type map 1912 that classifies different areas of the tissue as one of a tumor tissue type or a non-tumor tissue type. In various embodiments and without limitation, the processor 1906 determines whether the tissue types indicated in the tissue type map 1912 match expected tissue types at a target location 102. In various embodiments, the processor 1906 determines the tissue type map 1912 by comparing the impedance measurements 1908 to one or more characteristic impedance measurements associated with one or more tissue types. In various embodiments and without limitation, the processor 1906 determines whether the tissue type map 1912 indicates determined tissue types 1910 that match expected tissue types at a target location 102 (e.g., in order to determine whether the instrument head 108 is positioned at the target location). The method can return to step 2102 to record additional impedance measurements 1908 and to determine a second or updated tissue type map 1912.

In sum, the disclosed medical device measures the impedance of tissue in a location where an instrument head of a medical device is positioned. The medical device determines a tissue type map based on impedance measurements associated with two or more electrodes included in the instrument head. The disclosed approach advantageously results in the medical device determining a tissue type map of the tissue types at the location where the instrument head is located (e.g., without limitation, on various sides of the instrument head).

At least one technical advantage of the disclosed medical device relative to the prior art is that the disclosed medical device is able to determine a map of tissue types on one or more sides of the instrument head of a medical device prior to when a medical device tool is activated or during activation. For example, the disclosed medical device can determine whether the tissue types of portions of tissue located where the instrument head of a medical device is positioned match the expected tissue types at a given target location prior to activating the relevant medical device tool. In this manner, the disclosed medical device can ensure that medical device tools are applied to a selected tissue type, such as a tumor, rather than some other tissue types, such as healthy tissue. Also, the disclosed medical instrument can apply medical device tools at target locations more accurately than is possible with conventional medical devices. Consequently, the disclosed medical device can be used to perform various procedures, such as and without limitation, delivering therapeutic drugs or energy or extracting tissue samples, at specific locations more accurately and reliably than what can be achieved using conventional medical devices. These technical advantages provide one or more technological advancements over prior art approaches.

1. In some embodiments, a medical device comprises an instrument head that includes two or more electrodes and a medical device tool; an impedance bridge selectively coupled to the two or more electrodes; and a processor coupled to the impedance bridge.

2. The medical device of clause 1, wherein each of the two or more electrodes is shaped to curve outward relative to a longitudinal axis of the instrument head.

3. The medical device of clauses 1 or 2, wherein each of the two or more electrodes includes a flexible material.

4. The medical device of any of clauses 1-3, wherein each of the two or more electrodes comprises Nitinol.

5. The medical device of any of clauses 1-4, wherein the two or more electrodes extend to an adjustable extension length relative to an aperture of the instrument head.

6. The medical device of any of clauses 1-5, wherein the medical device tool includes a clamp, and the two or more electrodes are located at different positions along a length of the clamp.

7. The medical device of any of clauses 1-6, wherein the medical device tool includes a guidewire, and the two or more electrodes are located at different positions along a length of the guidewire.

8. The medical device of clause 7, wherein the instrument head includes a sheath, and the guidewire selectively retracts into the sheath.

9. The medical device of any of clauses 1-8, wherein the instrument head includes a sheath, the sheath includes an extension, and the guidewire selectively retracts into the extension.

10. The medical device of any of clauses 1-9, wherein the instrument head includes an extension, and the two or more electrodes are located at different positions along a length of the extension.

11. The medical device of clause 10, wherein the extension selectively bends at a bending location.

12. The medical device of clauses 10 or 11, wherein the extension selectively forms a curved shape.

13. The medical device of clause 12, wherein the extension selectively forms a first curved shape, a second curved shape, or a straight shape.

14. The medical device of any of clauses 10-13, wherein the extension selectively forms a circular shape that encircles a longitudinal axis of the instrument head.

15. The medical device of any of clauses 10-14, wherein the extension selectively forms a wave shape relative to a longitudinal axis of the instrument head.

16. The medical device of any of clauses 10-15, wherein the extension selectively forms a spiral shape.

17. The medical device of any of clauses 1-16, wherein the instrument head includes two or more guidewires, each guidewire extends from the instrument head in a different direction, and each of the two or more electrodes is located at a tip of a respective one of the two or more guidewires.

18. The medical device of any of clauses 1-17, wherein the instrument head includes two or more guidewires, each guidewire protrudes from the instrument head in a different outward direction, and each of the two or more electrodes is located at a lateral position along a respective one of the two or more guidewires.

19. The medical device of clause 18, wherein the two or more electrodes are arranged in a grid pattern.

20. The medical device of clauses 18 or 19, wherein the two or more guidewires are coupled to an extension tip of the instrument head, and retracting the extension tip changes a shape of each guidewire from a parallel configuration to a protruding configuration.

21. The medical device of any of clauses 1-20, wherein the instrument head includes two or more bands, each band protruding from the instrument head in different outward directions, each of the two or more electrodes is located at a lateral position along one of the two or more bands, the two or more bands are coupled to an extension tip of the instrument head, and retracting the extension tip changes a shape of each band from a parallel configuration to a protruding configuration.

22. The medical device of any of clauses 1-21, wherein the instrument head includes a balloon, a surface of the balloon includes two or more bands, each of the two or more electrodes is located at a lateral position along a respective one of the two or more bands, and inflating the balloon changes a shape of each band from a parallel configuration to a protruding configuration.

23. In some embodiments, a method for controlling medical device tools comprises recording, at one or more frequencies, two or more impedance measurements, wherein each impedance measurement is associated with two or more electrodes included in an instrument head of a medical device; and determining, based on the two or more impedance measurements, a tissue type map at a location associated with the instrument head.

24. The method of clause 23, wherein the two or more impedance measurements include a first impedance measurement associated with a first subset of the two or more electrodes and a second impedance measurement associated with a second subset of the two or more electrodes.

25. The method of clauses 23 or 24, wherein the two or more impedance measurements include a first impedance measurement associated with a first extension length of the two or more electrodes relative to an aperture of the instrument head and a second impedance measurement associated with a second extension length of the two or more electrodes relative to the aperture of the instrument head.

26. The method of any of clauses 23-25, wherein the two or more impedance measurements include a first impedance measurement taken at a first point in time and a second impedance measurement taken at a second point in time.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A medical device, comprising:
an instrument head that includes:
two or more electrodes, and
a medical device tool;
an impedance bridge selectively coupled to the two or more electrodes; and
a processor coupled to the impedance bridge.

2. The medical device of claim 1, wherein each of the two or more electrodes is shaped to curve outward relative to a longitudinal axis of the instrument head.

3. The medical device of claim 1, wherein each of the two or more electrodes includes a flexible material.

4. The medical device of claim 1, wherein each of the two or more electrodes comprises Nitinol.

5. The medical device of claim 1, wherein the two or more electrodes extend to an adjustable extension length relative to an aperture of the instrument head.

6. The medical device of claim 1, wherein the medical device tool includes a clamp, and the two or more electrodes are located at different positions along a length of the clamp.

7. The medical device of claim 1, wherein the medical device tool includes a guidewire, and the two or more electrodes are located at different positions along a length of the guidewire.

8. The medical device of claim 7, wherein the instrument head includes a sheath, and the guidewire selectively retracts into the sheath.

9. The medical device of claim 7, wherein the instrument head includes a sheath, the sheath includes an extension, and the guidewire selectively retracts into the extension.

10. The medical device of claim 1, wherein the instrument head includes an extension, and the two or more electrodes are located at different positions along a length of the extension.

11. The medical device of claim 10, wherein the extension selectively bends at a bending location.

12. The medical device of claim 10, wherein the extension selectively forms a curved shape.

13. The medical device of claim 12, wherein the extension selectively forms a first curved shape, a second curved shape, or a straight shape.

14. The medical device of claim 10, wherein the extension selectively forms a circular shape that encircles a longitudinal axis of the instrument head.

15. The medical device of claim 10, wherein the extension selectively forms a wave shape relative to a longitudinal axis of the instrument head.

16. The medical device of claim 10, wherein the extension selectively forms a spiral shape.

17. The medical device of claim 1, wherein the instrument head includes two or more guidewires, each guidewire extends from the instrument head in a different direction, and each of the two or more electrodes is located at a tip of a respective one of the two or more guidewires.

18. The medical device of claim 1, wherein the instrument head includes two or more guidewires, each guidewire protrudes from the instrument head in a different outward direction, and each of the two or more electrodes is located at a lateral position along a respective one of the two or more guidewires.

19. The medical device of claim 18, wherein the two or more electrodes are arranged in a grid pattern.

20. The medical device of claim 18, wherein the two or more guidewires are coupled to an extension tip of the instrument head, and retracting the extension tip changes a shape of each guidewire from a parallel configuration to a protruding configuration.

21. The medical device of claim 1, wherein the instrument head includes two or more bands, each band protruding from the instrument head in different outward directions, each of the two or more electrodes is located at a lateral position along one of the two or more bands, the two or more bands are coupled to an extension tip of the instrument head, and retracting the extension tip changes a shape of each band from a parallel configuration to a protruding configuration.

22. The medical device of claim 1, wherein the instrument head includes a balloon, a surface of the balloon includes two or more bands, each of the two or more electrodes is located at a lateral position along a respective one of the two or more bands, and inflating the balloon changes a shape of each band from a parallel configuration to a protruding configuration.

23. A method for controlling medical device tools, the method comprising:
recording, at one or more frequencies, two or more impedance measurements, wherein each impedance measurement is associated with two or more electrodes included in an instrument head of a medical device; and
determining, based on the two or more impedance measurements, a tissue type map at a location associated with the instrument head.

24. The method of claim 23, wherein the two or more impedance measurements include a first impedance measurement associated with a first subset of the two or more electrodes and a second impedance measurement associated with a second subset of the two or more electrodes.

25. The method of claim 23, wherein the two or more impedance measurements include a first impedance measurement associated with a first extension length of the two or more electrodes relative to an aperture of the instrument head and a second impedance measurement associated with a second extension length of the two or more electrodes relative to the aperture of the instrument head.

26. The method of claim 23, wherein the two or more impedance measurements include a first impedance measurement taken at a first point in time and a second impedance measurement taken at a second point in time.
